(19) United States
(12) Patent Application Publication
(10) Pub. No.: US 2022/0022940 A1
(43) Pub. Date: Jan. 27, 2022

(54) DEVICES AND METHODS FOR TRANSCATHETER HEART VALVE LEAFLET MODIFICATION

(71) Applicant: Mayo Foundation for Medical Education and Research, Rochester, MN (US)

(72) Inventor: Mayra Guerrero, Rochester, MN (US)

(21) Appl. No.: 17/311,740

(22) PCT Filed: Jan. 6, 2020
(86) PCT No.: PCT/US2020/012301

§ 371 Date: Jun. 8, 2021

Related U.S. Application Data
(60) Provisional application No. 62/788,490, filed on Jan. 4, 2019.

(51) Int. Cl. A61B 18/14

(52) U.S. Cl. CPC A61B 18/1445; A61B 2018/00369

(57) ABSTRACT

This document describes minimally invasive surgical instruments and methods for their use. For example, this document describes devices and methods for transcatheter modification of mitral valve leaflets to reduce or prevent the potential for full or partial blockages of the left ventricular outflow tract by anterior displacement of the anterior leaflet of the native mitral valve.

### CROSS-REFERENCE TO RELATED APPLICATIONS

This application claims the benefit of U.S. Provisional Application Ser. No. 62/788,490, filed Jan. 4, 2019. The disclosure of the prior application is considered part of (and is incorporated by reference in) the disclosure of this application.

### BACKGROUND

### 1. Technical Field

This document relates to minimally invasive surgical instruments and methods for their use. For example, this document relates to devices and methods for transcatheter modification of mitral valve leaflets to reduce or prevent the potential for full or partial blockages of the left ventricular outflow tract by anterior motion of the anterior leaflet of the native mitral valve.

### 2. Background Information

Some therapies for treating mitral valve pathology either mitral regurgitation (MR) or mitral stenosis (MS), such as transcatheter mitral valve replacement (TMVR) may at times worsen other (non-mitral) existing pathologic conditions, or create new pathologic conditions. One of the conditions to be managed is left ventricular outflow tract (LVOT) obstruction, or creation of high LVOT pressure gradients. Some implementations of prosthetic valve systems may physically obstruct the LVOT, and some benefits of TMVR may thereby be dissipated or lost. Further, in some implementations of prosthetic valve systems, anterior displacement of the native mitral valve leaflet(s) may cause LVOT obstruction or the creation of high LVOT pressure gradients. For example, in some cases anterior displacement is the incursion of an anterior leaflet of the native mitral valve into the LVOT during systole and/or diastole. In some cases, systolic anterior motion (SAM) is the incursion of an anterior leaflet of the native mitral valve into the LVOT during systole.

When a TMVR prosthetic valve is implanted in a native mitral valve without removal or other restraint of the native valve leaflets, the anterior leaflet may be exposed to different flow conditions that may actually “pull” the anterior leaflet, via Bernoulli forces, toward and into the LVOT or it may simply be displaced anteriorly by the transcatheter mitral valve. If the anterior leaflet is drawn too far into the LVOT, there is risk of it significantly interfering with the outflow, creating a significant clinical concern. There is therefore a potential benefit associated with modification of mitral valve leaflets, in conjunction with TMVR procedures, to reduce or prevent the potential for full or partial blockages of the LVOT by anterior displacement of the anterior leaflet of the native mitral valve.

### SUMMARY

This document describes minimally invasive surgical instruments and methods for their use. For example, this document describes devices and methods for transcatheter modification of mitral valve leaflets to reduce or prevent the potential for full or partial blockages of the LVOT by anterior displacement of the anterior leaflet of the native mitral valve. The devices can also be used to modify the leaflets of other heart valves such as the aortic and tricuspid valves.

In one aspect, this disclosure is directed to a transcatheter heart valve leaflet cutter device that includes an elongate catheter shaft and a leaflet cutter. The leaflet cutter includes a first shaft attached to and extending distally from a distal end of the catheter shaft, a C-shaped member with a first end attached to a distal end of the first shaft, a second shaft attached to a second end of the C-shaped member, and an expandable member attached to and extending along an edge of the first shaft that opposed the second shaft. The second shaft extends proximally away from the C-shaped member and terminates at a free end. The second shaft includes a cutting edge extending along an edge of the second shaft that faces an opposing edge of the first shaft. The cutting edge includes a sharp edge for cutting a heart valve leaflet. The expandable member is reconfigurable between: (i) a low-profile configuration in which the expandable member is spaced apart from the cutting edge and (ii) and expandable configuration in which the expandable member contacts the cutting edge.

Such a transcatheter heart valve leaflet cutter device may optionally include one or more of the following features. A distal end portion of the catheter shaft may be steerable. The transcatheter heart valve leaflet cutter device may also include a balloon member attached to the distal end portion of the catheter shaft. The balloon member may be expandable transversely away from the catheter shaft in a direction that is opposite of a direction of the second shaft's position in relation to the first shaft. In some embodiments, at least portion of the cutting edge is configured to be electrically energized to burn tissue of the heart valve leaflet. The catheter shaft may define a lumen for slidably advancing the transcatheter heart valve leaflet cutter over a guidewire. A central portion of the C-shaped member may be a distal-most end of transcatheter heart valve leaflet cutter.

In another aspect, this disclosure is directed to a transcatheter heart valve leaflet cutter device includes: (i) an outer tube defining a first lumen and a slot along a distal end portion of the outer tube; (ii) an inner tube slidably disposed within the first lumen and defining a second lumen; (iii) a blade cable slidably disposed within the second lumen; (iv) a blade pivotably attached to a distal end of the blade cable; and (v) an actuator attached to a proximal end of the blade cable. The actuator is translatable in relation to the outer tube to cause the blade to move correspondingly in relation to the outer tube as the blade is extending transversely through the slot.

Such a transcatheter heart valve leaflet cutter device may optionally include one or more of the following features. The blade may be fully contained within the first lumen when the actuator is in a fully proximal position relative to the outer tube. The blade may be a semilunar shape blade with a sharp point for puncturing. The blade may include one or more RF electrodes. The blade may be biased to self-extend through the slot. The transcatheter heart valve leaflet cutter may also include a second actuator that is actuatable to cause the blade to extend through the slot.

In another aspect, this disclosure is directed to a method of cutting a mitral valve leaflet using any of the transcatheter heart valve leaflet cutter devices described herein. The method uses a transapical approach to deploy the transcatheter heart valve leaflet cutter into a region of the mitral valve leaflet.

Such a method may optionally include one or more of the following features. The mitral valve leaflet may be cut by pulling the actuator proximally. The mitral valve leaflet may be cut by pulling the actuator proximally along a track of a balloon or prosthetic mitral valve.

In another aspect, this disclosure is directed to another transcatheter heart valve leaflet cutter device. The transcatheter heart valve leaflet cutter device includes (a) an outer tube defining a first lumen and a slot along a distal end portion of the outer tube; (b) a blade shaft slidably disposed within the first lumen; (c) a blade pivotably attached to a distal end of the blade shaft; and (d) an actuator attached to a proximal end of the blade shaft. The actuator is translatable in relation to the outer tube to cause the blade to move correspondingly in relation to the outer tube as the blade is extending transversely through the slot.

Such a transcatheter heart valve leaflet cutter device may optionally include one or more of the following features. The blade may be fully contained within the first lumen when the actuator is in a fully proximal position relative to the outer tube. The blade may be a semilunar shape blade with a sharp point for puncturing. The blade may include one or more RF electrodes. The blade may be biased to self-extend through the slot. The transcatheter heart valve leaflet cutter may also include a second actuator that is actuatable to cause the blade to extend through the slot.

In another aspect, this disclosure is directed to a method of cutting a mitral valve leaflet using any of the transcatheter heart valve leaflet cutter devices described herein. The method uses a transseptal approach to deploy the transcatheter heart valve leaflet cutter into a region of the mitral valve leaflet.

Such a method may optionally include one or more of the following features. The mitral valve leaflet may be cut by pushing the actuator distally. The mitral valve leaflet may be cut by pushing the actuator proximally along a track of a balloon or prosthetic mitral valve.

Particular embodiments of the subject matter described in this document can be implemented to realize one or more of the following advantages. First, some embodiments of the transcatheter leaflet cutting devices described herein can be used in a completely percutaneous/transcatheter procedure that is safe, reliable, and repeatable by surgeons and/or interventional cardiologists of a variety of different skill levels. Such minimally invasive techniques can reduce recovery times, patient discomfort, and treatment costs.

Second, some embodiments of the transcatheter leaflet cutting devices described herein are configured to reduce or prevent the potential for a natural mitral valve anterior leaflet to “flop” outward and/or from being drawn by a Venturi effect into the LVOT. Accordingly, performing leaflet modifications using the transcatheter leaflet cutting devices can reduce the risk of full or partial blockages of the LVOT. In some patient scenarios, the potential for suffering future adverse health events, such as heart failure, is thereby reduced.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram showing a heart undergoing a TMVR procedure and a leaflet cutting procedure using a transcatheter leaflet cutting device in a transapical approach in accordance with some embodiments provided herein.

FIG. 2 is a depiction of an example transcatheter leaflet cutting device in accordance with some embodiments provided herein.

FIG. 3 is view of a distal tip portion of the transcatheter leaflet cutting device of FIG. 2 with the cutting blade in a deployed configuration.

FIG. 4 is a cross-sectional view of a distal end portion of the transcatheter leaflet cutting device of FIG. 2.

FIGS. 5-8 depict various configurations of another example transcatheter leaflet cutting device in accordance with some embodiments provided herein.

FIG. 9 is a schematic diagram showing a heart undergoing a leaflet cutting procedure using a transcatheter leaflet cutting device in a transseptal approach in accordance with some embodiments provided herein.

FIG. 10 is a view of a distal tip portion of another example transcatheter leaflet cutting device in accordance with some embodiments provided herein.

FIG. 11 is view of the distal tip portion of the transcatheter leaflet cutting device of FIG. 10 with the cutting blade in a first deployed position.

FIG. 12 is view of the distal tip portion of the transcatheter leaflet cutting device of FIG. 10 with the cutting blade in a second deployed position.

FIG. 13 is a view of a distal tip portion of another example transcatheter leaflet cutting device in accordance with some embodiments provided herein.

Like reference numbers represent corresponding parts throughout.

### DETAILED DESCRIPTION

This document describes minimally invasive surgical instruments and methods for their use. For example, this document describes devices and methods for transcatheter modification of mitral valve leaflets to reduce or prevent the potential for full or partial blockages of the LVOT by anterior displacement of the anterior leaflet of the native mitral valve. The devices and methods described herein can be used in conjunction with TMVR procedures, as described further below. Moreover, the devices and methods describe herein can be used in other contexts such that they are not limited to use solely in conjunction with TMVR procedures. For example, the devices and methods described herein can also be used for tricuspid valve leaflet modification and aortic valve leaflet modification.

As described further below, the devices and methods described for cutting an anterior leaflet of a native mitral valve can be used in at least two different percutaneous approaches to access the mitral valve region. Such percutaneous approaches are minimally invasive techniques (without open-heart surgery). One approach to the mitral valve region is transapical (e.g., via an intercostal incision). Another approach is to access the right atrium using a trans-vascular approach (e.g., via the femoral vein and into the inferior vena cava). From the right atrium the devices can be passed through a trans-septal puncture of the fossa ovalis to enter into the left atrium adjacent the mitral valve region. This approach can be referred to as a transseptal approach. An approach via the aortic valve is also envisioned. One non-limiting example of transcatheter mitral valve products can be a TENDYNE™ valve, other mitral valve products can be used for TMVR procedure as well.

In some implementations, the devices and methods described herein are used in conjunction with one or more imaging modalities such as x-ray fluoroscopy, echocardiography, magnetic resonance imaging, computed tomography (CT), and the like. Some embodiments of the transcatheter leaflet cutting devices described herein can include one or more features to enhance the visibility of the transcatheter leaflet cutting devices via the imaging modalities. For example, in some embodiments the transcatheter leaflet cutting devices can include one or more radiopaque markers that can be used to identify a position and/or orientation of the transcatheter leaflet cutting devices while using fluoroscopy. Various surgical and interventional tools and techniques, such as guidewires, guide catheters, endoscopic scopes and tools, robotic or tele-operated surgery systems, and the like, can also be used to assist with the use of the devices and performance of the methods described herein.

In some embodiments, the transcatheter leaflet cutting devices described herein deploy a blade (e.g., semilunar shape blade or other shape blade) that punctures the leaflet near the base of the anterior leaflet (e.g., near the mitral valve annulus). In some embodiments, the blade is deployed simultaneously with deployment of a transcatheter prosthetic mitral valve. In some embodiments, the blade is deployed before or after deployment of a transcatheter prosthetic mitral valve.

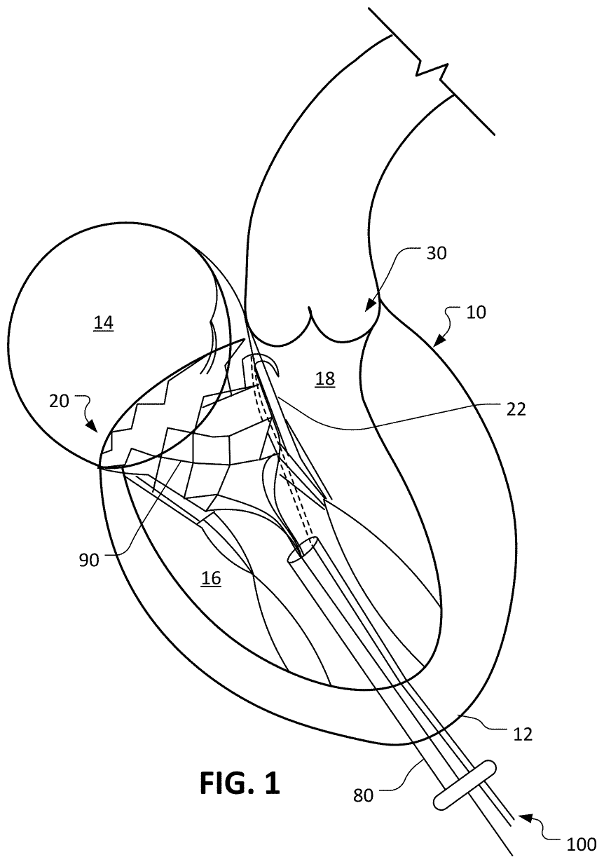

Referring to FIG. 1, a prosthetic mitral valve 90 can be implanted to replace the function of a native mitral valve 20 in a heart 10. The depicted TMVR procedure is using a transapical approach. That is, a sheath 80 is positioned through an apex 12 of the heart 10 and into the left ventricle 16. The heart 10 also includes a left atrium 14, a LVOT 18, and an aortic valve 30.

In the depicted embodiment used as an example in this illustration, the prosthetic mitral valve 90 is a self-expanding valve. That is, when the prosthetic mitral valve 90 emerges from its delivery catheter/sheath (which can be the sheath 80 or another catheter/sheath), the prosthetic mitral valve 90 will naturally tend to diametrically self-expand due to the shape memory of the framework of the prosthetic mitral valve 90. Alternatively, the prosthetic mitral valve 90 can be expandable by a balloon (i.e., balloon-expandable) in some embodiments.

An example transcatheter leaflet cutter 100 in accordance with some embodiments of this inventive disclosure is also depicted. In the depicted embodiment, the transcatheter leaflet cutter 100 is deployed through the transapical sheath 80 to access the region of the mitral valve 20. The transcatheter leaflet cutter 100 is used to make one or more cuts in the anterior leaflet 22 of the native mitral valve 20. By making such one or more cuts in the anterior leaflet 22, the potential for the anterior leaflet 22 to obstruct the LVOT 18 is reduced or eliminated because the anterior leaflet 22 will be splayed by the one or more cuts.

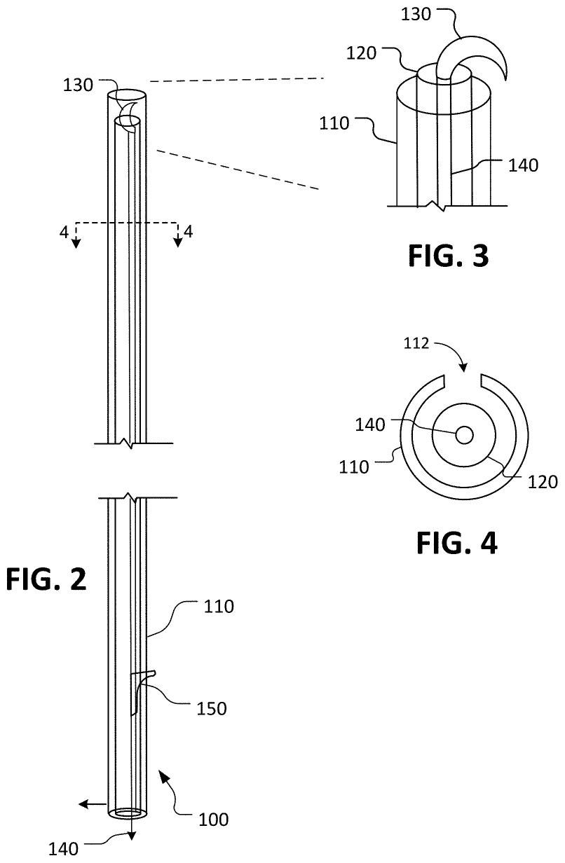

Referring also to FIGS. 2-4, the transcatheter leaflet cutter 100 includes an outer tube 110, an inner tube 120, a blade 130, a blade shaft 140, and an actuator 150. The outer tube 110 defines a first lumen in which the inner tube 120 is slidably disposed. The inner tube 120 defines a second lumen in which the blade 130 and blade shaft 140 are slidably disposed. The blade 130 is pivotably attached to the blade shaft 140. The actuator 150 is attached to the blade shaft 140 and translatable in relation to the outer tube 110. In some embodiments, the blade shaft 140 can be a cable.

The outer tube 110 and/or inner tube 120 can comprise a tubular polymeric or metallic material. For example, in some embodiments the outer tube 110 and/or inner tube 120 (and the tubes/catheters of the other transcatheter leaflet cutting devices described herein) can be made from polymeric materials such as, but not limited to, polytetrafluoroethylene (PTFE), fluorinated ethylene propylene (FEP), HYTREL®, nylon, PICOFLEX®, PEBAX® (e.g., 63D Pebax®), TECOFLEX®, and the like, and combinations thereof. In alternative embodiments, the outer tube 110 and/or inner tube 120 can be made from metallic materials such as, but not limited to, nitinol, stainless steel, stainless steel alloys, titanium, titanium alloys, and the like, and combinations thereof. In some embodiments, the outer tube 110 and/or inner tube 120 (and the tubes/catheters of the other transcatheter leaflet cutting devices described herein) can be made from combinations of such polymeric and metallic materials (e.g., polymer layers with metal braid, coil reinforcement, stiffening members, and the like, and combinations thereof). In some embodiments, one or more portions of the outer tube 110 and/or the inner tube 120 (and the tubes/catheters of the other transcatheter leaflet cutting devices described herein) can be steerable or deflectable in response to a clinician operator's control. Radiopaque markers can be included on any suitable portions of the outer tube 110 and/or inner tube 120 (and other parts of the transcatheter leaflet cutter 100).

The outer tube 110 includes a distal longitudinal slot 112 extending along its distal end portion (e.g., as shown in the cross-sectional view of FIG. 4). The distal longitudinal slot 112 allows clearance for the blade 130 to extend transversely outward from the outer wall surface or outer diameter of the outer tube 110 (in a cutting configuration as depicted in FIG. 3), and so the blade 130 can be translated proximally/distally parallel to the longitudinal axis of the transcatheter leaflet cutter 100 while the blade 130 is transversely extended to create a leaflet cut.

In some embodiments, such as the depicted embodiment, the blade 130 is a semilunar shape blade with a sharp point for puncturing. In particular embodiments, the blade 130 (and the blades or cutting edges of the other transcatheter leaflet cutting devices described herein) can include one or more radiofrequency (RF) electrodes to assist with puncturing and/or cutting the leaflet 22.

In some embodiments, the blade 130 transversely self-extends through the distal longitudinal slot 112 by virtue of shape memory, spring-bias, and/or other types of material/mechanical forces. In some embodiments, the blade 130 is manually controllable/actuatable (e.g., using the actuator 150, or one or more other manual actuation mechanisms) to extend/retract outward from the outer wall surface or outer diameter of the outer tube 110 (through the distal longitudinal slot 112). In particular embodiments, the second lumen of the inner tube 120 is used to contain and constrain the blade 130. Then, when the blade 130 is caused to emerge from the inner tube 120 the blade 130 will naturally pivot so as to extend outward from the outer wall surface or outer diameter of the outer tube 110 (through the distal longitudinal slot 112).

Still referring to FIG. 1, when the blade 130 is caused to extend outward from the outer wall surface or outer diameter of the outer tube 110 (through the distal longitudinal slot 112), the blade 130 will puncture through the anterior leaflet 22 as shown. After the blade 130 has punctured the anterior leaflet 22, then the blade shaft 140 can be pulled proximally so that the blade 130 will cut the anterior leaflet 22 from the puncture point all the way to the free edge of the anterior leaflet 22. In some embodiments, the process can be repeated to create two or more such cuts in the anterior leaflet 22.

In the depicted embodiment, while the prosthetic mitral valve 90 is separate from the transcatheter leaflet cutter 100 (they are not directly attached to each other in this embodiment), the prosthetic mitral valve 90 abuts against the transcatheter leaflet cutter 100 to provide some pressure against the transcatheter leaflet cutter 100 to assist the blade 130 to puncture and slit the anterior leaflet 22.

In some other embodiments, the prosthetic mitral valve 90 and the transcatheter leaflet cutter 100 are attached to each other. For example, in some embodiments the framework of the prosthetic mitral valve 90 can include a rail along which the blade 130 can travel.

In still other embodiments (as described further below), a balloon can be used to provide pressure against the transcatheter leaflet cutter 100 to assist the blade 130 to puncture and slit the anterior leaflet 22. The transcatheter leaflet cutter 100 can either be attached to the balloon or separate from the balloon (while abutting each other). In some such embodiments, the balloon is also used to deploy (radially expand) the prosthetic mitral valve 90.

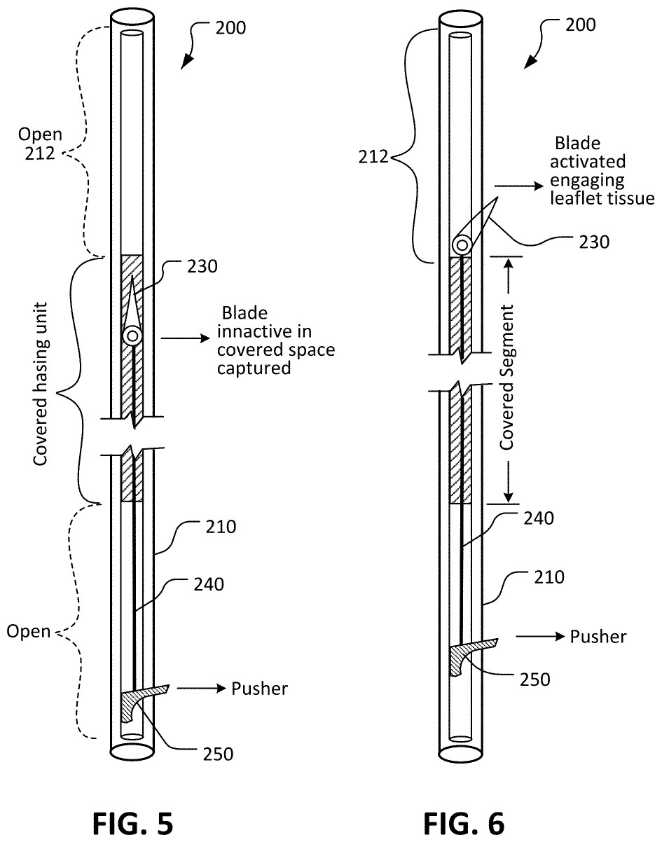

Referring to FIGS. 5-8, another example transcatheter leaflet cutter 200 in accordance with some embodiments of this inventive disclosure is depicted. In this series of figures, the transcatheter leaflet cutter 200 is depicted in various configurations or arrangements corresponding to the use of the transcatheter leaflet cutter 200, as described further below.

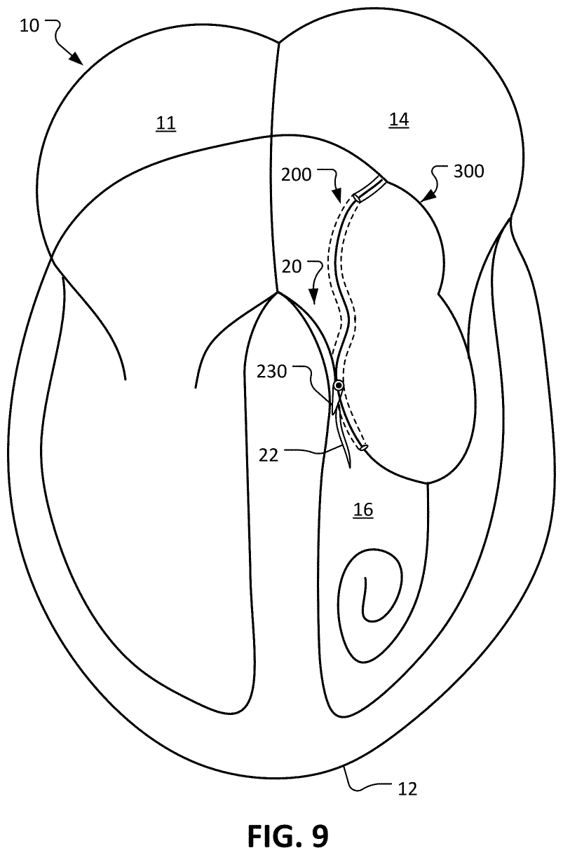

This transcatheter leaflet cutter 200 is designed for cutting the anterior leaflet of a native mitral valve using a transseptal approach. That is, as described further below in reference to FIG. 9, the transcatheter leaflet cutter 200 is designed to cut as its blade 230 is being pushed distally (as opposed to the transcatheter leaflet cutter 100 described above that is designed to cut as its blade 130 is being pulled proximally).

In the configuration of FIG. 5, the transcatheter leaflet cutter 200 is in a delivery/retrieval configuration. The blade 230 is inactive and fully contained within a covered portion of the outer tube 210. In this configuration, the transcatheter leaflet cutter 200 can be navigated within the patient's vasculature to/from the region of the patient's native mitral valve without the risk of inadvertent tissue damage from the blade 230 (since it is not exposed).

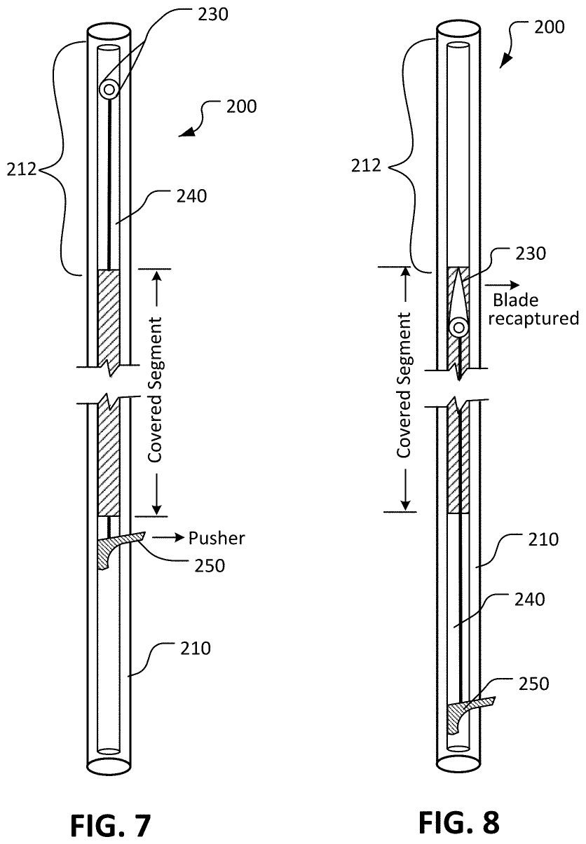

In the configuration of FIGS. 6 and 7, a clinician operator has manually moved the actuator 250 to push the blade shaft 240 and blade 230 distally. As described above in reference to outer tube 110, the outer tube 210 has a distal longitudinal slot 212 extending along its distal end portion. The blade 230 will extend radially outward through the distal longitudinal slot 212 either naturally or by being manually actuated to do so. When the blade 230 emerges through the distal longitudinal slot 212 it can puncture the native anterior leaflet near the base of the leaflet. Then, as the clinician operator pushed the actuator 250 distally, the blade 230 will cut leaflet tissue along an inferior direction (i.e., from the native mitral valve toward the apex of the heart).

In the configuration of FIG. 8, the actuator 250 has been pulled back into its starting position (after the leaflet cutting has been performed) so that the blade 230 is once again fully contained within the covered portion of the outer tube 210. In this configuration, the transcatheter leaflet cutter 200 can be removed from the patient's vasculature without the risk of inadvertent tissue damage from the blade 230 (since it is not exposed).

Referring to FIG. 9, an example transseptal approach (from the right atrium 11 to the left atrium 14) using the transcatheter leaflet cutter 200 and a balloon 300 is depicted. While it is not required in all embodiments, in this example the transcatheter leaflet cutter 200 and the balloon 300 are integrated/attached. That is, the balloon 300 includes a distal end portion of the outer tube 210, or includes a track along which the blade 230 will travel during its distally-extending cutting process.

As shown, in some embodiments the balloon 300 can have an hourglass shape with a waist region that can be positioned approximately at the annulus of the native mitral valve 20. In some embodiments, the balloon 300 can also be used to expand a prosthetic mitral valve in situ.

In the depicted embodiment, the transcatheter leaflet cutter 200 is deployed and used to make one or more cuts in the anterior leaflet 22 of the native mitral valve 20. By making such one or more cuts in the anterior leaflet 22, the potential for the anterior leaflet 22 to obstruct the LVOT is reduced or eliminated because the anterior leaflet 22 will be splayed by the one or more cuts.

In some embodiments, the transcatheter leaflet cutter 200 can be attached to the transcatheter prosthetic mitral valve and be removed at the end of the TMVR procedure. Once the transcatheter prosthetic mitral valve is deployed, the blade 230 is activated and inserted in the anterior mitral leaflet 22. Then the blade is advanced distally/inferiorly toward the apex 12 while cutting the anterior leaflet 22 (as depicted by FIGS. 6 and 7). Once cutting has been completed, the blade 230 is retracted (as depicted by FIG. 8) and transcatheter leaflet cutter 200 is removed from the patient through the patient's vasculature.

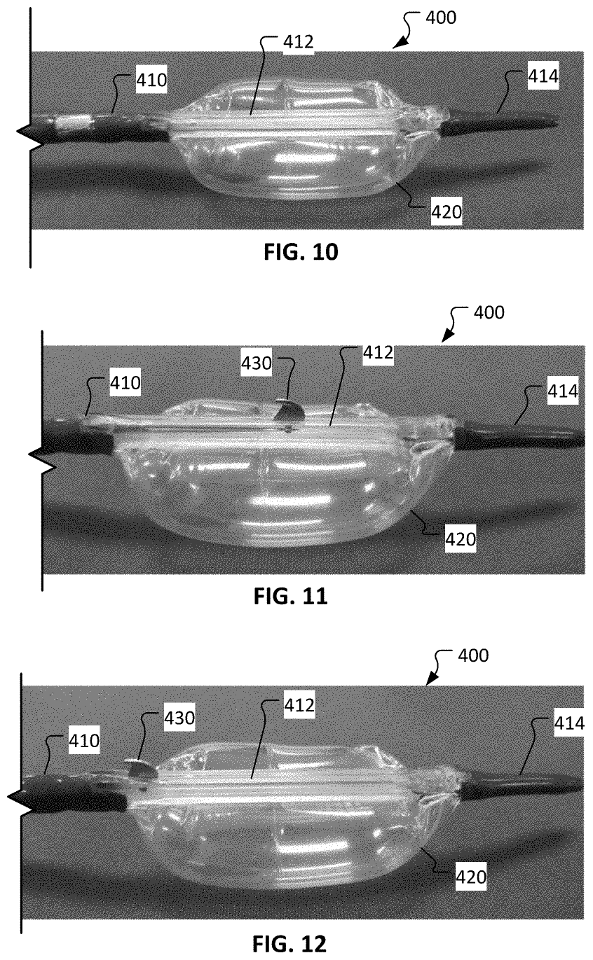

FIGS. 10-12 show another transcatheter leaflet cutting device in accordance with some embodiments described herein. In particular, the transcatheter leaflet cutter 400 includes an elongate catheter 410, a balloon member 420, and a blade 430.

The transcatheter leaflet cutter 400 can be deployed in any suitable manner (e.g., transseptal, transapical, trans-aortic, etc.). The transcatheter leaflet cutter 400 can be delivered within a sheath and/or over a guidewire in some implementations. When positioned in the area of the target heart valve, the clinician user can control and actuate the transcatheter leaflet cutter 400 to make one or more cuts to the native leaflet tissue.

The catheter 410 includes a track portion 412 and a distal end portion 414. The track portion 412 defines an open slot that the blade 430 can radially extend from and longitudinally travel along.

The balloon member 420 is attached to the catheter 410. In the depicted embodiment, the balloon member 420 is attached on an opposite side of the catheter 410 in comparison to the open slot of the track portion 412. The balloon member 420 is configured to be expanded radially outward from a side of the catheter 410, radially away from the side of the catheter 410 that defines the open slot of the track portion 412. In some embodiments, the balloon member 420 has a generally D-shaped cross-sectional shape. Accordingly, the balloon member 420 is configured to provide backpressure for the blade 430 to do its cutting. For example, in some implementations the balloon member 420 can be positioned adjacent to an annulus of a heart valve, and then the balloon member 420 can be expanded. The balloon member 420 can thereby force the track portion 412 against a leaflet of the heart valve. Thereafter, the blade 430 can be radially deployed and linearly actuated to cut the leaflet.

The blade 430 is extendable radially outward through the longitudinal slot defined by the track portion 412, either naturally (e.g., shape-memory) or by being manually actuated to do so. When the blade 430 emerges through the longitudinal slot of the track portion 412, the blade 430 can puncture the native anterior leaflet near the base of the leaflet. Then, as the clinician operator pushes an actuator distally, or draws an actuator proximally, the blade 430 will cut leaflet tissue along a path corresponding to the longitudinal slot defined by the track portion 412 (e.g., from the base of the leaflet near the annulus of the valve, toward the edge of the leaflet).

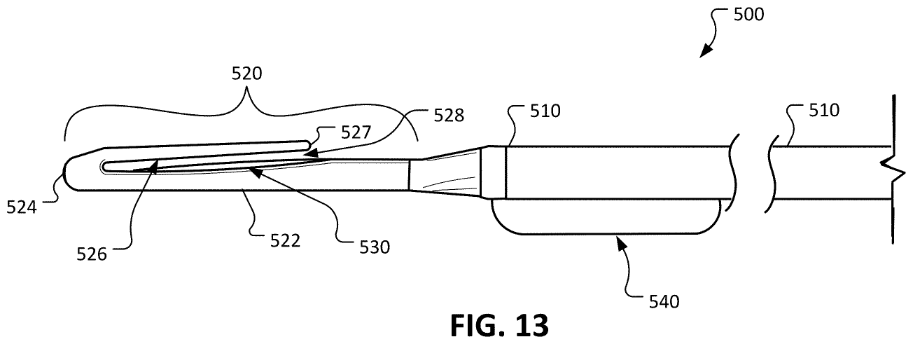

FIG. 13 shows another transcatheter leaflet cutting device in accordance with some embodiments described herein. In particular, the transcatheter leaflet cutter 500 includes an elongate catheter 510 and a leaflet cutter 520. In some embodiments, an optional balloon member 540 is included.

The transcatheter leaflet cutter 500 can be deployed in any suitable manner (e.g., transseptal, transapical, trans-aortic, etc.). The transcatheter leaflet cutter 500 can be delivered within a sheath and/or over a guidewire in some implementations. When positioned in the area of the target heart valve, the clinician user can control and actuate the transcatheter leaflet cutter 500 to make one or more cuts to the leaflet tissue.

In some embodiments, one or more portions of the catheter 510 are steerable by a clinician operator using an actuator handle. The optional balloon member 540 is attached to a distal end portion of the catheter 510. The balloon member 540 is expandable, transversely or radially away from the catheter shaft 510.

The leaflet cutter 520 is attached to a distal end of the shaft of the catheter 510. The leaflet cutter 520 comprises a first shaft 522 attached to and extending distally from a distal end of the catheter shaft 510. The leaflet cutter 520 also comprises a C-shaped member 524 with a first end attached to a distal end of the first shaft 522. The leaflet cutter 520 also comprises a second shaft 526 attached to a second end of the C-shaped member 524.

The second shaft 526 extends proximally, away from the C-shaped member 524, and terminates at a free end 527. Accordingly, the leaflet cutter 520, with its arrangement of the first shaft 522 and the second shaft 526 that are adjoined at the middle C-shaped section 524, makes up a hook-shape. This arrangement defines an elongate narrow space 528 extending between the first shaft 522 and the second shaft 526 in which leaflet tissue is received in preparation for cutting the leaflet tissue.

The second shaft 526 includes a cutting edge extending along an internal edge of the second shaft 526. The cutting edge comprises a sharpened, knife-edge configured for cutting tissue of a heart valve leaflet. The cutting edge of the second shaft 526 faces an opposing edge of the first shaft 522. That is, the cutting edge of the second shaft 526 makes up one side of the elongate narrow space 528 extending between the first shaft 522 and the second shaft 526 in which leaflet tissue is received.

An expandable member 530 (shown here in its low-profile configuration) is attached to and extending along the first shaft 522. In some embodiments, the expandable member 530 is a high-pressure balloon. In some embodiments, the expandable member 530 can be a mechanical member that can be actuated to move it transversely outward from the first shaft 522 toward the cutting edge of the second shaft 526. Being on an opposite side of the narrow space 528 in comparison to the cutting edge of the second shaft 526, the expandable member 530 can make up at least a portion of the first shaft's side of the elongate narrow space 528 between the first shaft 522 and the second shaft 526 in which leaflet tissue is received.

The expandable member 530 is reconfigurable between: (i) a low-profile configuration in which the expandable member 530 is spaced apart from the cutting edge of the second shaft 526 and (ii) and expandable configuration in which the expandable member extends transversely outward from the first shaft 522 and contacts the cutting edge of the second shaft 526.

The expandable member 530 provides force against the leaflet tissue within the narrow space 528 to force the tissue against the cutting edge of the second shaft 526. Accordingly, the leaflet can be severed as a result of being forced against the cutting edge of the second shaft 526 by the expandable member 530. In some embodiments, the optional balloon member 540, which extends transversely outward from the catheter 510 in a direction that is opposite of a direction of the position of the second shaft 526 in relation to the first shaft 522, can also provide a retaining force to help stabilize the leaflet cutter 520 during use.

In some embodiments, at least portion of the cutting edge extending along the internal edge of the second shaft 526 is configured to be electrically energized to burn tissue of the heart valve leaflet. For example, in some embodiments one or more RF electrodes can be positioned along the cutting edge extending along the internal edge of the second shaft 526. The electrodes can be electrified to assist the leaflet cutting action of the leaflet cutter 520.

In some embodiments, the catheter shaft 510 defines a lumen for slidably advancing the transcatheter heart valve leaflet cutter 500 over a guidewire.

As shown in FIG. 13, in some embodiments, a central portion of the C-shaped member 524 is a distal-most end of transcatheter heart valve leaflet cutter 500. The distal-most central portion of the C-shaped member 524 can be radiused, as shown, to provide an atraumatic tip for the transcatheter heart valve leaflet cutter 500.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A transcatheter heart valve leaflet cutter, comprising:
an elongate catheter shaft; and
a leaflet cutter comprising:
a first shaft attached to and extending distally from a distal end of the catheter shaft;
a C-shaped member with a first end attached to a distal end of the first shaft;
a second shaft attached to a second end of the C-shaped member, the second shaft extending proximally away from the C-shaped member and terminating at a free end, wherein the second shaft comprises a cutting edge extending along an edge of the second shaft that faces an opposing edge of the first shaft, wherein the cutting edge comprises a sharp edge for cutting a heart valve leaflet; and
an expandable member attached to and extending along the opposing edge of the first shaft, the expandable member being reconfigurable between: (i) a low-profile configuration in which the expandable member is spaced apart from the cutting edge and (ii) and expandable configuration in which the expandable member contacts the cutting edge.

2. The transcatheter heart valve leaflet cutter of claim 1, wherein a distal end portion of the catheter shaft is steerable.

3. The transcatheter heart valve leaflet cutter of claim 1, further comprising a balloon member attached to the distal end portion of the catheter shaft, wherein the balloon member is expandable transversely away from the catheter shaft in a direction that is opposite of a direction of the second shaft's position in relation to the first shaft.

4. The transcatheter heart valve leaflet cutter of claim 1, wherein at least portion of the cutting edge is configured to be electrically energized to burn tissue of the heart valve leaflet.

5. The transcatheter heart valve leaflet cutter of claim 1, wherein the catheter shaft defines a lumen for slidably advancing the transcatheter heart valve leaflet cutter over a guidewire.

6. The transcatheter heart valve leaflet cutter of claim 1, wherein a central portion of the C-shaped member is a distal-most end of transcatheter heart valve leaflet cutter.

7. A transcatheter heart valve leaflet cutter, comprising:
an outer tube defining a first lumen and a slot along a distal end portion of the outer tube;
an inner tube slidably disposed within the first lumen and defining a second lumen;
a blade cable slidably disposed within the second lumen;
a blade pivotably attached to a distal end of the blade cable; and
an actuator attached to a proximal end of the blade cable, the actuator translatable in relation to the outer tube to cause the blade to move correspondingly in relation to the outer tube as the blade is extending transversely through the slot.

8. The transcatheter heart valve leaflet cutter of claim 7, wherein the blade is fully contained within the first lumen when the actuator is in a fully proximal position relative to the outer tube.

9. The transcatheter heart valve leaflet cutter of claim 7, wherein the blade is a semilunar shape blade with a sharp point for puncturing.

10. The transcatheter heart valve leaflet cutter of claim 7, wherein the blade includes one or more RF electrodes.

11. The transcatheter heart valve leaflet cutter of claim 7, wherein the blade is biased to self-extend through the slot.

12. The transcatheter heart valve leaflet cutter of claim 7, further comprising a second actuator that is actuatable to cause the blade to extend through the slot.

13-15. (canceled)

16. A transcatheter heart valve leaflet cutter, comprising:
an outer tube defining a first lumen and a slot along a distal end portion of the outer tube;
a blade shaft slidably disposed within the first lumen;
a blade pivotably attached to a distal end of the blade shaft; and
an actuator attached to a proximal end of the blade shaft, the actuator translatable in relation to the outer tube to cause the blade to move correspondingly in relation to the outer tube as the blade is extending transversely through the slot.

17. The transcatheter heart valve leaflet cutter of claim 16, wherein the blade is fully contained within the first lumen when the actuator is in a fully proximal position relative to the outer tube.

18. The transcatheter heart valve leaflet cutter of claim 16, wherein the blade is a semilunar shape blade with a sharp point for puncturing.

19. The transcatheter heart valve leaflet cutter of claim 16, wherein the blade includes one or more RF electrodes.

20. The transcatheter heart valve leaflet cutter of claim 16, wherein the blade is biased to self-extend through the slot.

21. The transcatheter heart valve leaflet cutter of claim 16, further comprising a second actuator that is actuatable to cause the blade to extend through the slot.

22-24. (canceled)
